(19) United States
(12) Patent Application Publication
(10) Pub. No.: US 2026/0066092 A1
(43) Pub. Date: Mar. 5, 2026

(54) ANGIOGRAPHY IMAGE GENERATION AND DISPLAY USING MULTIPLE MACHINE LEARNING MODELS

(71) Applicants: William E. BUTLER, Bristol, RI (US); AngioWave Imaging, Inc., Concord, MA (US)

(72) Inventors: William E. BUTLER, Bristol, RI (US); Hector ANDRADE-LOARCA, Munich (DE)

(21) Appl. No.: 19/315,041

(22) Filed: Aug. 29, 2025

Related U.S. Application Data
(60) Provisional application No. 63/689,308, filed on Aug. 30, 2024.

(51) Int. Cl. G16H 30/20

(52) U.S. Cl. CPC G16H 30/20

(57) ABSTRACT

Systems, methods, and computer program products for generating and displaying angiography images from multiple machine learning models that provide different sensitivity and specificity performance in the segmentation of vascular structures in an angiogram. A graphical user interface with control elements provides user control over the mixture of the multiple machine learning models and other settings, including an a pseudo brightness control and a zoom control. The displayed angiogram image is based on the settings of the control elements, and adjusts in response to changes thereto. The pseudo brightness control controls the mixture of the high specificity model versus the high sensitivity model in the displayed image. The zoom control modifies the magnification of the displayed image and increases the proportional mixture of the high sensitivity model in the displayed image. A widget or other type of control element may also be provided for controlling the mixture of the empirical data versus machine learning model output in the displayed image.

### CROSS-REFERENCE TO RELATED APPLICATIONS

This application claims priority to U.S. Provisional Patent Application No. 63/689,308, filed Aug. 30, 2024 and titled DISPLAY OF ANGIOGRAPHY IMAGES FROM PARAMETERIZED DEEP LEARNING MODELS, which is incorporated by reference herein in its entirety.

### FIELD OF THE INVENTION

The present invention relates generally to systems, methods, and computer program products for generating and displaying angiographic images using multiple machine learning models.

### BACKGROUND OF THE INVENTION

The heart sends blood to the body as a sequence of arterial stroke volumes. The pumped blood crosses the capillaries to the veins and returns to the heart. The presence and motion of blood in the blood vessels can be dynamically imaged with a technique called angiography. In fluoroscopic x-ray angiography, a chemical contrast agent that attenuates x-rays greater than blood or tissue is injected into the vascular system in coordination with a sequence of x-ray images. The chemical (typically iodinated) contrast travels through the vasculature, blocks the passage of the x-rays at a given frame rate, and this spatiotemporal x-ray attenuation pattern creates a sequence of images on an x-ray sensor. These angiographic images are typically two dimensional in space and one dimensional in time.

In standard practice, the image quality of the angiographic images in a study can be improved by increasing the dose of the injected chemical contrast and/or by increasing the dose of the fluoroscopic x-ray radiation. Such dosage increase(s) can increase the risk of harm to the patient or subject due to toxic side effects of the chemical contrast agent and/or x-ray radiation. For example, the chemical contrast agent can be toxic to the kidneys and produce a significant mass load on the vascular system, which can stress the heart and other vascular structures. Higher x-ray doses can directly injure various bio-molecular constituents of tissue, including those composed by DNA in a cell's nuclear apparatus, which can lead to malformations of organs and a potential risk of neoplasia.

U.S. patent application Ser. No. 18/192,439 to Butler, filed Mar. 29, 2023 and titled SYSTEM AND METHOD FOR ANGIOGRAPHIC DOSE REDUCTION USING MACHINE LEARNING (Butler), U.S. Provisional Patent Application No. 63/586,141, filed on Sep. 28, 2023 and titled SYSTEM AND METHOD FOR ANGIOGRAPHIC DOSE REDUCTION USING MACHINE LEARNING WITH A CONCORDANCE METRIC, and U.S. patent application Ser. No. 18/899,118, filed on Sep. 27, 2024 and titled SYSTEM AND METHOD FOR ANGIOGRAPHIC DOSE REDUCTION USING MACHINE LEARNING WITH A CONCORDANCE METRIC, which patent applications are hereby incorporated by reference herein in their entireties, describe the use of neural network models to reduce the radiation or chemical contrast dose while maintaining image quality. However, the machine learning models focus on optimizing either sensitivity or specificity, but not both. Focusing on one or the other can lead to a trade-off between detecting as many relevant features in the image as possible and minimizing detection of irrelevant features in the image.

### SUMMARY OF THE INVENTION

A first aspect of the invention is a method for generating and displaying angiographic images on a display connected to a computer. In certain embodiments, the method comprises obtaining, with the computer, a first angiographic image generated by a first machine learning model from angiographic data and a second angiographic image generated by a second machine learning model from the angiographic data, wherein the first machine learning model is configured to have greater sensitivity performance than the second machine learning model, and wherein the second machine learning model is configured to have greater specificity performance than the first model; and displaying, with the computer via the display, a mixture of the first and second angiographic images generated by the first and second machine learning models. By displaying a mixture of the images generated by different machine learning models, such embodiments provide a new technical capability that can improve the accuracy and reliability of angiography image analysis.

In certain embodiments, the first angiographic image comprises a first set of pixels and the second angiographic image comprises a second set of pixels, wherein displaying the mixture of the first and second angiographic images comprises superposing the first and second sets of pixels. In certain embodiments, the method further comprises adjusting, with the computer, the mixture of the first and second angiographic images by increasing or decreasing an opacity of one of the first and second sets of pixels relative to an opacity of the other of the first and second sets of pixels. Such embodiments facilitate rapid combination of the first and second angiographic images for display by the computer.

In certain embodiments, the method may also comprise receiving, with the computer, an input from a user, and adjusting the mixture of the first and second angiographic images based on the input from the user. Such embodiments facilitate angiography image analysis by allowing the user to select a mixture that displays information most useful to the user.

In certain embodiments, the input from the user is received from a user-adjustable control element connected to the computer, the control element has a range of adjustment, at one end of the range, an opacity of the first set of pixels is greater than an opacity of the second set of pixels, and at another end of the range, the opacity of the second set of pixels is greater than the opacity of the first set of pixels. The control element may be configured as a mechanical control element, a graphical control element, or a combination of both. Such embodiments facilitate angiography image analysis by providing an easily understood and efficient way to interface with the computer in order to adjust what is displayed.

In certain embodiments, the method may further comprise displaying, with the computer via the display, a graphical user interface comprising the graphical control element. The graphical control element may be adjustable via a pointing device connected to the computer. The displaying may further comprise mixing “original” or “raw” or “empirical” angiographic image data from an imaging device with first and second angiographic images generated from the raw angiographic image data by first and second machine learning models trained to optimize different aspects of angiography image analysis (e.g., in the case of neural network models, using different loss functions optimized for sensitivity and specificity, respectively). The method may also comprise adjusting, with the computer, a mixture of the raw angiographic image data and the first and second angiographic images based on an input from a user received by the computer. The displaying may further comprise adjusting a zoom level of an image. Such embodiments facilitate angiography image analysis by allowing the user to adjust the information that is displayed.

Another aspect of the invention is a method of training machine learning models for enhanced visualization of angiographic images. In certain embodiments, neural network models are used, and a loss function is supplied to each neural network model during training. The loss function can be optimized for high sensitivity of blood vessel structures. Alternatively, the loss function may be optimized for high specificity for the detection of blood vessel structures. The result of training with a specific loss function is a parameterized neural network model. In certain embodiments, a first parametrized neural network model is trained for high sensitivity, and a second parameterized neural network model is trained for high specificity. In other embodiments, a combined neural network model may be trained for high sensitivity and high specificity. For example, a combined neural network model may be trained to output two images: one optimized for high sensitivity and another optimized for high specificity.

The machine learning models can be any type of suitable machine learning models, such as convolutional neural network models, deep neural network models, deep convolutional neural network models, vision transformer (ViT) models, or support vector machine (SVM) models. In certain embodiments, all of the machine learning models comprise the same type of machine learning model. In other embodiments, at least one of the machine learning models is a first type of machine learning model and at least one of the other machine learning models is a second type of machine learning model that is different than the first type of machine learning model. In certain embodiments, when a neural network model for detecting blood vessels is trained, the training software is supplied with a loss function. The properties of the loss function influence the sensitivity of the technique for detecting spatial temporal vascular structures.

In another aspect of the invention, a system is provided. In certain embodiments, the system comprises a computer with one or more processors, a display, and a non-transitory computer-readable medium coupled to the one or more processors and storing instructions thereon that, when executed by the one or more processors, causes the one or more processors to perform one or more of the methods described herein.

In another aspect of the invention, a computer program product is provided. In certain embodiments, the computer program product comprises a non-transitory computer-readable medium storing instructions thereon that, when executed by one or more processors, causes the one or more processors to perform one or more of the methods described herein.

In another aspect of the invention, a graphical user interface for an angiographic display system is provided that comprises an arrangement of one or more graphical control elements or widgets controllable by a user to combine the outputs of multiple machine learning models, each optimized for different aspects of angiography image analysis (e.g., in the case of a neural network model, by being trained with different loss functions). In certain embodiments, the graphical user interface may also include one or more graphical control elements or widgets controllable by the user to combine the raw angiographic image from an imaging device with a mixture of images from the multiple machine learning models. In certain embodiments, the graphical user interface may also include one or more graphical control elements or widgets controllable by the user to provide a zoom feature that expands a selected portion of a displayed image. In certain embodiments, the graphical control elements or widgets are displayed on a display connected to the computer and controllable by the user via a user input device (e.g., a keyboard, a trackpad, a touchscreen, a mouse, or combinations thereof) connected to the computer. Such techniques can allow users to easily adjust and optimize the display of angiographic image data.

Other features and advantages of the invention will be apparent from the specification and drawings.

### BRIEF DESCRIPTION OF THE DRAWINGS

For a more complete understanding of the invention, reference is made to the following description and accompanying drawings, in which:

FIG. 1A shows a screen shot of a graphical user interface with user-adjustable control elements according to a first embodiment, wherein the control elements are set to particular settings to display a raw angiogram image;

FIG. 1B shows a screen shot of a graphical user interface with user-adjustable control elements according to the first embodiment, wherein the control elements are shown with particular settings to display a segmented image a generated by a machine learning model trained for maximum specificity;

FIG. 1C shows a screen shot of a graphical user interface with user-adjustable control elements according to the first embodiment, wherein the control elements are shown with particular settings to display a segmented image generated by a machine learning model trained for maximum sensitivity;

FIG. 2A shows a screen shot of a graphical user interface with user-adjustable control elements according to a second embodiment, wherein the control elements are set to particular settings to display the raw angiogram image;

FIG. 2B shows a screen shot of a graphical user interface with user-adjustable control elements according to the second embodiment, wherein the control elements are set to particular settings to display a segmented image generated by a machine learning model trained for maximum specificity, and with panning applied thereto;

FIG. 2C shows a screen shot of a graphical user interface with user-adjustable control elements according to the second embodiment, wherein the control elements are set to particular settings to display a segmented image which is enlarged, has the same panning position as the image in FIG. 2B, and a higher contribution of information from a machine learning model trained for high sensitivity;

FIG. 3 is a partially schematic view of an example of a rotational x-ray system that may be used with embodiments of the disclosure for acquiring angiographic data;

FIG. 4 is an additional partially schematic view of an example of a rotational x-ray system that may be used with embodiments of the disclosure for acquiring angiographic data;

FIG. 5 is a schematic diagram of a general computer system or information processing device that may be used with embodiments of the disclosure;

FIG. 6 is a schematic diagram of general computing environment that may be used with embodiments of the disclosure; and

FIG. 7 is a flowchart of an example methodology, in accordance with an embodiment of the invention.

### DETAILED DESCRIPTION OF THE PREFERRED EMBODIMENTS

Systems, methods, and computer program products are provided for combining the outputs of multiple machine learning models trained to optimize different attributes of angiographic images (e.g., specificity and sensitivity). In certain embodiments, the machine learning models are neural network models trained with different weighted loss functions. For example, one neural network model may be trained with a weighted loss function optimized for specificity, and another neural network model may be trained with a weighted loss function optimized for sensitivity. Each image frame of an angiogram from an x-ray imaging device may be inputted to the trained machine learning models to generate multiple segmented angiographic images of the image frame in which different attributes (e.g., specificity or sensitivity) are optimized. As used herein, a segmented angiographic image is an angiographic image in which pixels corresponding to blood vessels are identified and delineated from surrounding pixels. As used herein, the term “specificity” refers to a model's ability to correctly identify pixels in an image that are not part of an object of interest (e.g., not part of a blood vessel), whereas the term “sensitivity” refers to a model's ability to correctly identify pixels in an image that are part of the object of interest (e.g., part of a blood vessel). A model with high specificity will produce fewer false identifications of the object of interest in an image than a model with high sensitivity, while the model with high sensitivity will correctly identify more objects of interest in an image than the model with high specificity.

The enhanced angiographic images generated by the machine learning models and the raw images from the imaging device may be displayed on a display connected to a computer or the imaging device. In certain embodiments, a mixture of the angiographic images generated by the machine learning models may be displayed, and the angiographic images generated by the machine learning models may optionally be mixed with raw angiographic image data from the imaging device. The mixtures displayed to the user on the display may be controlled by the user via one or more graphic user interfaces. To enhance intuitive use, graphical user interface widgets (e.g., graphical elements that a user can interact with to provide an input to the graphical user interface) may be provided as part of the one or more graphical user interfaces. The widgets may resemble familiar metaphors from everyday objects, and be designed to simulate a brightness control for a display (e.g., to adjust an amount of specificity), a zoom control (e.g., magnification), a model control (e.g., to adjust the portion of the image to display together with the amount of sensitivity), and various control combinations thereof. In other example embodiments, physical hardware controls (such as separate dials, knobs, or sliders) may be provided for controlling the mixture of angiographic images generated by the machine learning models, and/or controlling the mixture of machine learning model-generated angiographic images with raw angiographic image data (sometimes referred to herein as emphical angiographic data or empirical data).

The machine learning models can be any type of suitable machine learning models, such as convolutional neural network models, deep neural network models, deep convolutional neural network models, ViT models, or SVM models. In certain embodiments, all of the machine learning models comprise the same type of machine learning model. In other embodiments, at least one of the machine learning models is a first type of machine learning model and at least one of the other machine learning models is a second type of machine learning model that is different than the first type of machine learning model. An example of a deep neural network model suitable for use with the disclosed embodiments is U-Net (see Ronnenberger et al, “U-Net: Convolutional Networks for Biomedical Image Segmentation, Lecture Notes in Computer Science: Medical Image Computing and Computer-Assisted Intervention,” 2015, pp 234-241, incorporated by reference herein).

In certain embodiments, when training a machine learning model (such as a U-Net neural network model) for the spatiotemporal segmentation of angiography data, a so-called “label” image and a continuous sequence of angiographic image frames are supplied. The label image represents ground truth (e.g., a correctly segmented image) against which a machine learning models's predictions are compared when learning. The label image may be drawn by hand by a human expert while inspecting the corresponding angiographic images. As described by U.S. patent application Ser. No. 18/192,439 to Butler and U.S. Provisional Patent Application No. 63/586,141 (both of which are incorporated by reference above), the angiographic image frame of interest can be in the middle of a sequence of angiographic image frames. This arrangement allows the machine learning model to learn the spatiotemporal structure of angiographic data. In one embodiment, the angiographic sequence consists of five frames, and the frame of interest (the target label frame) is the middle (e.g., third) frame.

In one embodiment of the invention, the spatial dimensions are represented by (512×512) pixels. In the label image, the background pixels have a value of ‘0’ and the vessel pixels have a value of ‘1’. A typical angiogram has more background pixels than vessel pixels. The ratio is typically about 15:1 background to vessel pixels.

### Cross-Entropy Loss Function

When computing a cross-entropy loss function L, the pixels may be enumerated from 1 to N=512×512=262,144 total pixels. For the i^{th} pixel, the label is symbolized with yᵢ and the predicted label at a given training round with [symbol] . For the training iteration, the cross-entropy error function may be defined as shown in Equation (1):

$$( y , \hat{y} ) = - \frac{1}{N} {\sum }_{i = 1}^{N} [ {y}_{i} log ( {\hat{y}}_{i} ) + ( 1 - {y}_{i} ) log ( 1 - {\hat{y}}_{i} ) ] \qquad ( 1 )$$

Equation (1) assumes an equal number of background and vessel pixels in the label image. Assuming, on average, W_{b} background pixels and Wᵥ vessel pixels, the weighted cross-entropy is given by Equation (2):

$$( y , \hat{y} , {W}_{v} , {W}_{b} ) = - \frac{1}{N} {\sum }_{i = 1}^{N} [ {W}_{v} {y}_{i} log ( {\hat{y}}_{i} ) + {W}_{b} ( 1 - {y}_{i} ) log ( 1 - {\hat{y}}_{i} ) ] \qquad ( 2 )$$

A ratio of Wᵥ:W_{b}=10 biases the loss function against sensitivity to vessel pixels. A ratio of Wᵥ:W_{b}=20 biases the loss function toward a greater sensitivity for vessel pixels. Upon training, two different models may be obtained. The prior model (Wᵥ:W_{b}=10) is optimized for high specificity (model Mₚ) to vessel pixels and the latter model (Wᵥ:W_{b}=20) is optimized for high sensitivity (model Mₛ). These models can be used to analyze the same angiographic image data and produce two different segmentation results.

While the above-described loss function is one example, those skilled in the art will recognize and appreciate that other loss functions may be used to produce parameterized neural network models. Similarly, while the above example can employ a UNet variant, which can have an encoder-decoder structure and skip connections, it will be appreciated that other deep learning variants may be used to similar effect.

### Merging Image Data

Using two models, Mₚ and Mₛ, that are respectively tuned by differently parameterized loss functions during training will produce two sets of predictions, ŷₚ and ŷₛ, from the same empirical data, ‘y’, in the inference stage of model execution. Thus, three separate image sources can be merged into a single image in the graphical user interface, based on y, ŷₚ, ŷₛ. As further discussed below, in accordance with various embodiments of the invention, these three data sources may be used to generate a single flexible image that may be displayed in a graphical user interface that further comprises one or more widgets that are controllable by a user via a user input device connected to the computer to adjust certain attributes of the image. For example, embodiments of the invention allow for the merging of raw data, y, and segmented data, ŷ, derived from an angiographic study. In particular, the relative proportion of a given image data source (such as Mₚ and Mₛ) on image generation may be based on user input, e.g., via user-controlled widgets (further discussed below).

### Merging Image Data

As discussed above, segmented angiographic images generated by the machine learning models and the raw images from the imaging device may be displayed on a display connected to a computer or the imaging device. In certain embodiments, a mixture of the angiographic images generated by the machine learning models may be displayed, and the angiographic images generated by the machine learning models may optionally be mixed with raw angiographic image data from the imaging device. The mixtures displayed to the user on the display may be controlled by the user via one or more graphical user interfaces.

In certain embodiments, the graphical user interface(s) may comprise graphical user interface widgets (also referred to herein as ‘widgets,’ ‘widget displays,’ ‘graphical user interface widget displays,’ and ‘control elements’) that may be utilized as angiography display controls for merging multiple images produced in accordance with these differing models (e.g., Mₚ and Mₛ). The graphical user interface widgets can be of various sizes and shapes, and may be based on familiar objects, symbols, metaphors, and the like.

In certain embodiments, the image data for y, ŷₚ, ŷₛ can be merged and used to generate a single display image on a computer monitor or display by attaching controls of the alpha channel to widgets on the graphical user interface that rely on familiar metaphors. In other words, widgets in the graphical user interface may be utilized which allow users to adjust images onscreen in accordance with desired parameter changes (zoom, panning, degree or weight of machine learning model specificity influence, degree or weight of machine learning model sensitivity influence, etc.).

A pixel in a displayed image may conventionally be considered to be composed of four numbers, also known as channels. Three of the channels may represent colors in the RGB color model. For example, the three numbers may represent red, green, and blue, and each number may have a different intensity value. Combining these values using the different intensities of each allows for the representation of millions of different colors. In particular, the color of a pixel from across the visible spectrum may be synthesized by the comparative value of each of these individual numbers. In angiography, images are generally in grayscale, not in color. As a result, an angiographic image typically has two channels, a grayscale channel and an alpha channel. While exemplary embodiments illustrated and described herein employ two channels, it will be appreciated that such embodiments and other embodiments of the invention may also be used for the generation and display of color images represented by four channels.

The alpha channel represents the opacity of a pixel in an image. It is based on the metaphor of a ray trace (e.g., line of sight) from the eye of the viewer to two images on a computer display monitor superimposed on each other. The two images superimposed on each other are imagined to be stacked one on top of the other so that if the image closer to the eye of the viewer has an alpha channel value in each pixel of one, then each pixel is completely opaque, and the further image is not rendered or viewed. Conversely, if the pixels of the image closer to the viewer have a value in the alpha channel of zero, then that image closer to the eye of the viewer is not displayed at all. Instead, the viewer sees solely the image that is further away from the viewer. Intermediate values of the alpha channel between zero and one allow for intermediate mixtures of the two images.

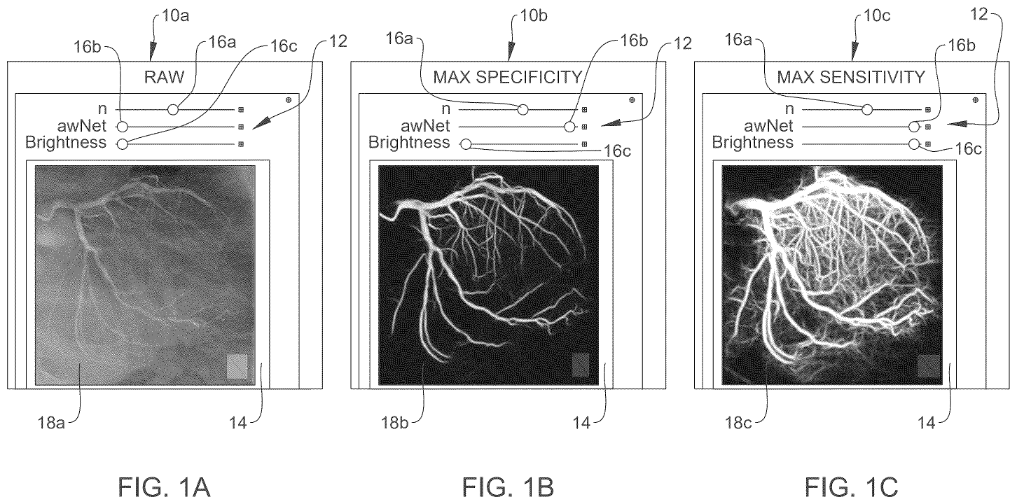

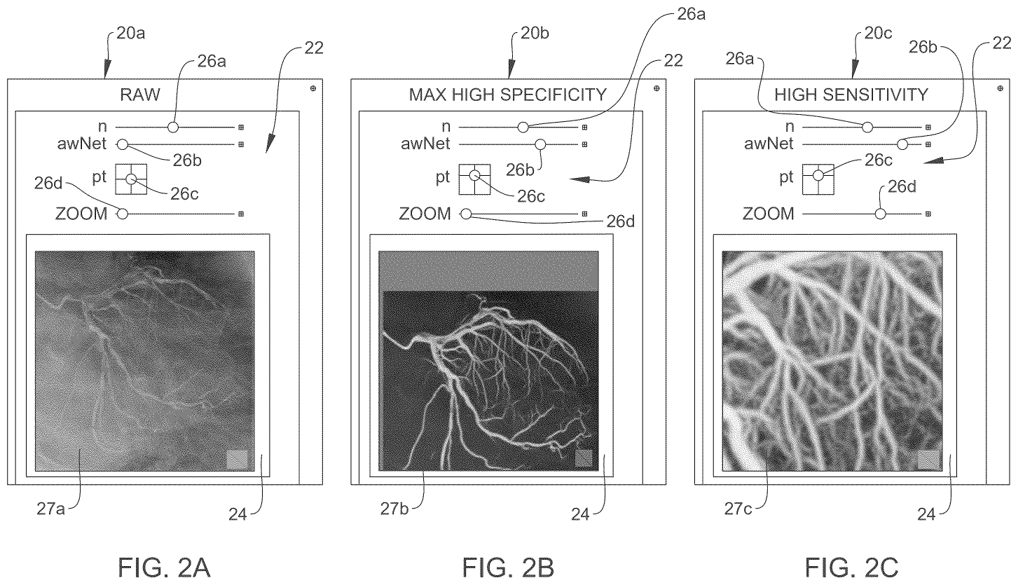

Embodiments of the present invention may be based on two alpha channels, α_{d} and αₘ, adjustable by a user via interactive widgets in the graphical user interface. The alpha channel α_{d}, may control, for example, the particular mixture of the empirical data y with the two predicted segmentation model, data ŷₚ and ŷₛ. An example widget for adjusting alpha channel α_{d} is labeled AWNet in FIGS. 1A-2C. Even though the predicted data ŷₚ and ŷₛ emerge from two separately trained models, Mₚ and Mₛ, respectively; for simplicity in the graphical user interface, they may be represented by this particular widget as one model, e.g., the inventor's AWNet neural network model. In other embodiments, separate widgets may be used to allow a user to control the weights of the image from each predicted model separately. In certain embodiments, three or four separately trained models may be utilized, and each may be assigned a corresponding user-controllable widget on a graphical user interface, whereby a user may be able to control the relative weight/strength/mixture of influence of each model. An illustration of the use of these widgets to achieve these results is further discussed below with respect to FIGS. 1A-2C.

The alpha channel, αₘ, controls the relative mixture in the representation of the two trained models, Mₚ and Mₛ. On the display screen, the image data can be thought of as being layered from farthest (e.g., distal) to nearest (e.g., proximal) from the human user in the order y, yₚ, and yₛ. The rendering scheme (RS) may be represented as Equation (3):

$$RS = y + {α}_{d} ( {\hat{y}}_{p} + {α}_{m} {\hat{y}}_{s} ) \qquad ( 3 )$$

By way of example, if αₘ is set to 1 at every pixel, then only ŷₛ is displayed. If αₘ is set to 0 and if α_{d} is set to 1 at every pixel, then only ŷₚ is displayed. If both αₘ and od are set to 0 at every pixel, then only the empirical data y is displayed. Such scenarios are delineated below in Table-1:

TABLE 1

| αₘ | α_{d} | Dataset Displayed |
|---|---|---|
| 0 | 0 | y |
| 0 | 0 < α_{d} < 1 | y + α_{d}(ŷₚ + αₘŷₛ) |
| 0 | 1 | ŷₚ |
| 0 < αₘ < 1 | 0 | y + α_{d}(ŷₚ + αₘŷₛ) |
| 0 < αₘ < 1 | 0 < α_{d} < 1 | y + α_{d}(ŷₚ + αₘŷₛ) |
| 0 < αₘ < 1 | 1 | (ŷₚ + αₘŷₛ) |
| 1 | 0 | ŷₛ |
| 1 | 0 < α_{d} < 1 | ŷₛ |
| 1 | 1 | ŷₛ |

A separate rendering scheme (RS2), represented as Equation (4):

(4) RS2 = (1 − α_{d})y + α_{d}((1 − αₘ)ŷₚ + αₘŷₛ) where {α_{d}, αₘ ∈[0,1]}

is another formulation to represent how α_{d} and αₘ control the relative mixture in the representation of the two trained models, Mp and Ms.

In certain embodiments, two graphical user interface widget arrangements may be utilized. A first widget may be a brightness control element based on a brightness metaphor, and a second widget may be a zoom control element based on a zoom or closeness to an image metaphor. These control elements may or may not be used simultaneously. The brightness control element may be, for example, a physical knob that a user turns or a widget displayed on a graphical user interface screen that a user controls with an input device such as a computer mouse, a trackpad, or a touch screen connected to a computer. In certain embodiments, increasing a brightness setting increases the representation of the model parameterized for further sensitivity, M. Increasing the brightness does not necessarily increase the actual luminance of the display monitor, but appears to do so in a metaphorical sense. A user may change the brightness setting by interacting with mechanical knobs, buttons, levers, and the like, or by interacting with a graphical user interface displayed via computer display software, to change the αₘ setting.

In certain embodiments, a zoom control element (physical and/or software-based) may be provided that enables a user to zoom in on (enlarge/magnify) and/or move around (pan) an image. Increasing the amount of zoom via the control element increases the magnification of the image surrounding the region of panning interest and causes a modification to the αₘ parameter to increase the proportional representation of Mₛ. As the image is enlarged, if the outer image boundaries exceed a display port on a computer monitor (or within a graphical user interface displayed on a computer monitor), the image may be accordingly cropped to fit within the same display port. An example display port has dimensions of 512 by 512 pixels. Thus, two control elements may be utilized for controlling the αₘ parameter, one based on a brightness metaphor, and the other based on a zooming/magnification metaphor.

An angiogram consists of a sequence of image frames. The particular image frame that is displayed on the two-dimensional screen may be controlled by a separate e control element, such as a physical knob or a software-based control element such as a graphical widget. In certain embodiments, systems described herein may establish a relationship between a frame selector control element and the α_{d} and αₘ selection control elements, and allow a user to modify/control the α_{d} and αₘ control elements. The user's selections may be maintained (e.g., kept constant) as the image frame selector control element is adjusted.

### Display of Angiographic Images

FIGS. 1A-1C show screen shots 10a-c of a graphical user interface 12 with a display window 14 and control elements 16a-c for rendering an angiographic image from raw image data and a mixture of image data generated by parameterized neural network models, in accordance with certain embodiments of the present invention. The screen shots 10a-c in FIG. 1 show the same graphical user interface 12 displaying the same image frame of the same angiogram but with control elements 16a-c of the graphical user interface being set differently in each screen shot to display the raw image 18a or a model-generated image 18b or 18c with different degrees of specificity and sensitivity, respectively.

The control elements of the graphical user interface 12 shown in FIGS. 1A-1C include an image frame selector widget 16a for selecting an image frame ‘n’ of the angiographic study to display, a raw image vs. model-generated image mixture control widget 16b to adjust a mixture of the raw image with a model-generated image, and a model-generated image mixture control widget 16c for adjusting a mixture of images generated by the models. Widget 106c is also referred to herein as a brightness control because increasing the prevalence of the image generated by the model trained to optimize sensitivity relative to the model trained to optimize specificity may create a perception of increased brightness even though an intensity of pixels may not change.

In the example embodiment of FIGS. 1A-C, the control elements 16a-c are shown as graphical control elements (or widgets) in the form of sliders. Each slider includes a knob or button that may be dragged left or right to adjust (e.g., to decrease or increase) an assigned value. The graphical control elements 16a-c in the first screen shot 10a are shown adjusted to display the raw angiogram image 18a. In the second screen shot 10b, the control elements 16a-c are shown adjusted to display only an image 18b generated by the neural network model trained for maximal specificity; and, in the third screen shot 10c, the control elements 16a-c are shown adjusted to display only an image generated by the neural network model trained for maximal sensitivity.

In each of the screen shots 10a-c, the control element 16a is adjusted to the same image frame about halfway through the angiographic study. Thus, each of the images 18a-c represents a different rendering of the same image frame. In other words, by adjusting the control elements 16b-c to different settings, different renderings of the same image frame may be produced.

Referring now to FIG. 1A, the control element 16b (raw image vs. model-generated image) is set to display only the raw image 18a. That is, the control element 16b is adjusted all the way to the left, which causes only the raw image 18a to be displayed. If the control element 16b were adjusted all the way to the right, only a model-generated image would be displayed; and, if the control element 16b were adjusted somewhere in between left and right ends of the slider bar, a mixture of the raw image and a model-generated image would be displayed. The closer to the left the control element 16b is adjusted, the more prevalent the raw image is displayed relative to the model-generated image; and the closer to the right, the more prevalent the model-generated image is displayed relative to the raw image. There is also a brightness control element 16c set to show the more specific deep learning rendering (least bright). However, in this configuration (i.e., screen shot 10a), the brightness control element 16c has no effect because the raw versus model-generated control element 16b has been set to show only a raw image, whereas the brightness control element 16c controls only a mixture of model-generated images. Since no model-generated image is shown in screenshot 10a, how the brightness control element 16c is set is irrelevant

Referring to FIG. 1B, the control element 16b (raw image vs. model-generated image) is set to maximum (i.e., to only show an image produced by a neural network), and the brightness control element 16c is set to minimum (not bright) to produce an image mixture 18b with maximal specificity (i.e., to only show an image generated by the neural network model trained to provide maximal specificity). That is, the control element 16b is set all the way to the right (maximum amount of model-generated image), and the control element 16c is set all the way to the left (minimum brightness/amount of sensitivity model). In comparison with the raw image, the model-generated image is a segmented image clearly identifying blood vessels that could not easily be seen in the raw image. The user is thus able to focus in on portions of the image that may have been missed only looking at the raw image.

Referring to FIG. 1C, the control element 16b (raw image vs. model-generated image) is again set to maximum (i.e., to only show an image produced by a neural network); however, the brightness control element 16c is set to maximum brightness (all the way to the right) to produce an image mixture 18c with maximal sensitivity (i.e., to only show an image generated by the neural network model trained to provide maximal sensitivity). In comparison with the model-generated image 18b, the model-generated image 18c shows even more segmented blood vessels, thereby providing additional information to the user, albeit with less specificity.

FIGS. 2A-2C show screen shots 20a-c of another graphical user interface 22 with a display window 24 and control elements 26a-d for rendering an angiographic image from raw image data and a mixture of image data generated by parameterized neural network models, in accordance with certain embodiments of the present invention. The control elements of the graphical user interface 22 shown in FIGS. 2A-2C includes pan and zoom widgets 26c and 26d, respectively, in addition to an image frame selector widget 26a and a raw image vs. model-generated image mixture control widget 26b for rendering an angiographic image from a mixture of the raw image data and data generated by parameterized neural network models, in accordance with embodiments of the present invention. The screen shots in FIGS. 2A-2C show the same image frame of the same angiogram with control elements 26a-d of the graphical user interface 22 set differently in each screen shot to display the image frame with different degrees of specificity and sensitivity. More specifically, in each of the screen shots 20a-c, the control element 26a (image frame selector widget) is adjusted to the same image frame about halfway through the angiographic study. Thus, each of the images 27a-c represents a different rendering of the same image frame.

Referring still to FIGS. 2A-2C, a control element 26b (similar to control element 16b in FIGS. 1A-1C) is utilized along with zoom and panning control elements 26d and 26c to increase/decrease zooming and to pan to different sections of the image, respectively. Control element 26d (zoom) also controls the contribution of information from the high sensitivity model, eliminating the need for a brightness control element. It will be appreciated, however, that a separate brightness control element (like control element 16c) could be utilized in the graphical user interface 22 of FIGS. 2A-2C.

In the screenshot 20a shown in FIG. 2A, graphical control elements 26b-d are set to display the raw angiogram image 27a. In the screenshot 20b shown in FIG. 2B, the control elements 26b-d are set to display an image 27b that is a combination of the raw image and a model-generated image generated by the high specificity model, with some panning also applied to the image. In the screenshot 20c in FIG. 2C, the control elements 26b-d are set to display the same panning position as FIG. 2B, but with a higher zoom factor, and coordinated higher contribution of information from the high sensitivity model.

Referring now to FIG. 2A, the frame selector control element 26a is shown as a slider set to a position between left and right ends of the scroll bar, thus selecting an angiogram image from about halfway through the angiographic study. Raw versus neural network selector control element 26b is shown as a slider set all the way to the left on of the scroll bar so as to display only the raw image 27a in the display window 24 of graphical user interface 22. Zoom selector control element 26d is shown as a slider set all the way to the left on the scroll bar in order to not produce any zooming of image 27a within display window 24 of graphical user interface 22. Pan selector control element 26c is shown as a knob or button in a rectangular field representing the image. The knob or button may be translated in x and y directions within the rectangular field to select a section of the image to display. If the control element 26c is centered in the rectangular field, the entire image is displayed. If the control element 26c is translated away from the center of the rectangular field, only the section of the image corresponding to the position of the control element in the rectangular field is displayed. Pan control element 26c in FIG. 2A is centered, so there has been no panning of image 27 by the user in this example. This configuration provides no contribution from either the high specificity model or the high sensitivity model.

Referring now to FIG. 2B, the frame selector control element 26a is set the same as in FIG. 2A, but the raw versus neural network selector control element 26b is set to about ⅔ of maximum to show a blend of the raw image and information from one or more neural network models, and zoom control element 26d is set to minimum (i.e., at the far left of the scroll bar) to not produce any zooming of image 27b and to select information only from the high specificity model. Pan selector control element 26c has been moved upward relative to its location in FIG. 2A. The effect of these settings in graphical user interface 22 is a segmented angiographic image 27b with high specificity that is panned upwardly.

In FIG. 2C, the frame selector control element 26a, the raw versus neural network model control element 26b, and the pan selector control element 26c are set similarly as in FIG. 2B. However, zoom selector control element 26d is shifted to the right along the horizontal scroll bar. This causes a coupled increase in magnification and an increased contribution of the high sensitivity net model, thus generating a segmented angiographic image 27c of higher sensitivity that is also zoomed-in.

In the example embodiments depicted in FIGS. 1A-2C, display of the control elements of the graphical user interfaces above the image display window allows the control elements to be separately enumerated and viewed by the user. However, it will be appreciated that the control elements described herein may appear in other forms, positions, and orientations on screen. Additionally or alternatively, control elements described herein may be based on direct manipulation of physical control elements. For example, instead of a two-dimensional slider widget to control brightness, a middle finger roll wheel and a computer mouse may be utilized. Instead of a two-dimensional slider widget to control panning, in certain embodiments, a user may employ a mouse and the left click button to drag the image. Instead of a widget to control zoom, a user may press the shift key and rotate the middle finger mouse wheel to apply the zoom metaphor. In certain embodiments, the control elements/widgets may be physical knobs, sliders, and/or buttons configured to adjust the degree of specificity, sensitivity, zoom, image number, panning, etc., in the image panel displayed. In other embodiments, a combination of graphical control elements (e.g., widgets) and physical control elements (e.g., knobs, sliders, buttons, and the like) may be utilized.

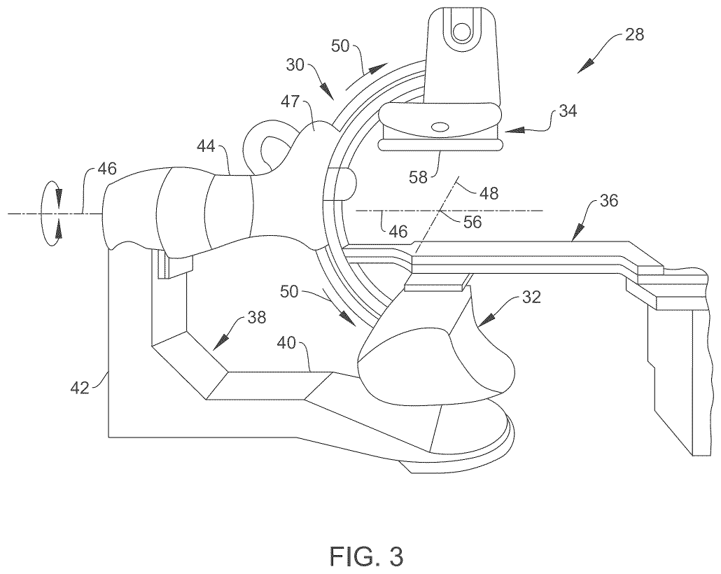

Referring to FIGS. 3, 4, and 5, exemplary systems or devices that may be employed for carrying out embodiments of the invention are illustrated. It will be understood that such systems and devices are only exemplary of representative systems and devices, and that other hardware and software configurations are suitable for use with embodiments of the invention. Thus, the embodiments are not intended to be limited to the specific systems and devices illustrated herein, and it will be recognized that other suitable systems and devices can be employed without departing from the spirit and scope of the subject matter provided herein.

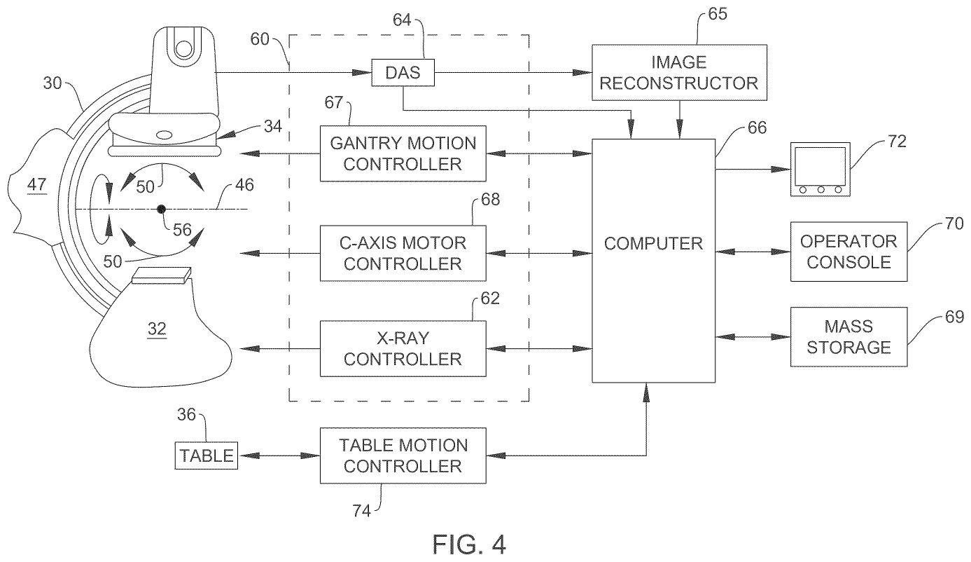

Referring first to FIGS. 3 and 4, a rotational x-ray system 28 is illustrated that may be employed for obtaining an angiogram at faster than cardiac rate, such as via fluoroscopic angiography. In acquiring an angiogram, a chemical contrast agent may be injected into the patient positioned between an x-ray source and detector, and x-ray projections are captured by the x-ray detector as a two-dimensional image (i.e., an angiographic image frame). A sequence of such image frames comprises an angiographic study or angiogram, and, in some embodiments of the invention, the angiographic image frames may be acquired at faster than cardiac frequency to facilitate vessel measurement.

As shown in FIG. 3, an example of an angiogram imaging system in the form of a rotational x-ray system 28 includes a gantry having a C-arm 30 which carries an x-ray source assembly 32 on one of its ends and an x-ray detector array assembly 34 at its other end. The gantry enables x-ray source assembly 32 and x-ray detector array assembly 34 to be oriented in different positions and angles around a patient disposed on a table 36, while providing to a physician access to the patient. The gantry includes a pedestal 38 which has a horizontal leg 40 that extends beneath table 36 and a vertical leg 42 that extends upward at the end of horizontal leg 40 that is spaced apart from table 36. A support arm 44 is rotatably fastened to the upper end of vertical leg 42 for rotation about a horizontal pivot axis 46.

Horizontal pivot axis 46 is aligned with the centerline of table 36, and support arm 44 extends radially outward from the horizontal pivot axis 46 to support a C-arm drive assembly 47 on its outer end. The C-arm 30 is slidably fastened to the C-arm drive assembly 47 and is coupled to a drive motor (not shown) which slides the C-arm 30 to revolve about a C-axis 48 as indicated by arrows 50. The horizontal pivot axis 46 and C-axis 48 intersect each other, at a system isocenter 56 located above table 36, and are perpendicular to each other.

X-ray source assembly 32 is mounted to one end of C-arm 30 and x-ray detector array assembly 34 is mounted to its other end. X-ray source assembly 32 emits a beam of x-rays which are directed at x-ray detector array assembly 34. Both assemblies 32 and 34 extend radially inward toward horizontal pivot axis 46 such that the center ray of this beam passes through system isocenter 56. The center ray of the beam thus can be rotated about system isocenter 56 around either horizontal pivot axis 46, C-axis 48, or both, during the acquisition of x-ray attenuation data from a subject placed on table 36.

X-ray source assembly 32 contains an x-ray source which emits a beam of x-rays when energized. The center ray passes through system isocenter 56 and impinges on a two-dimensional flat panel digital detector 58 housed in x-ray detector array assembly 34. The two-dimensional flat panel digital detector 58 may be, for example, a 2048×2048 element two-dimensional array of detector elements. Each element produces an electrical signal that represents the intensity of an impinging x-ray, and hence the attenuation of the x-ray as it passes through the patient. During a scan, x-ray source assembly 32 and x-ray detector array assembly 34 are rotated about system isocenter 56 to acquire x-ray attenuation projection data from different angles. In example devices, the detector array may be able to acquire a given number of (e.g., up to fifty) projections, or image frames, per second. The rate of image frames per second may determine how many image frames can be acquired for a prescribed scan path and speed.

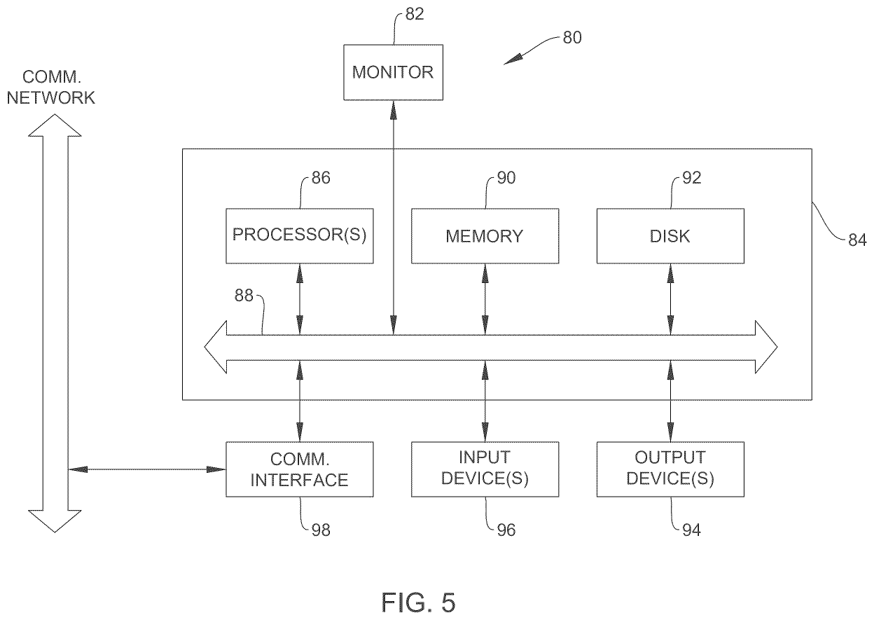

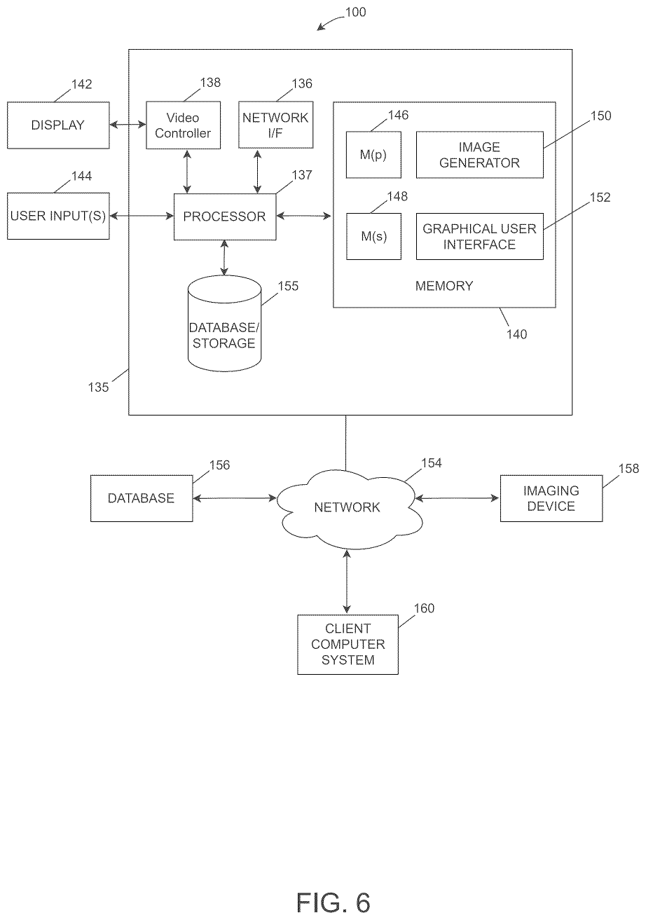

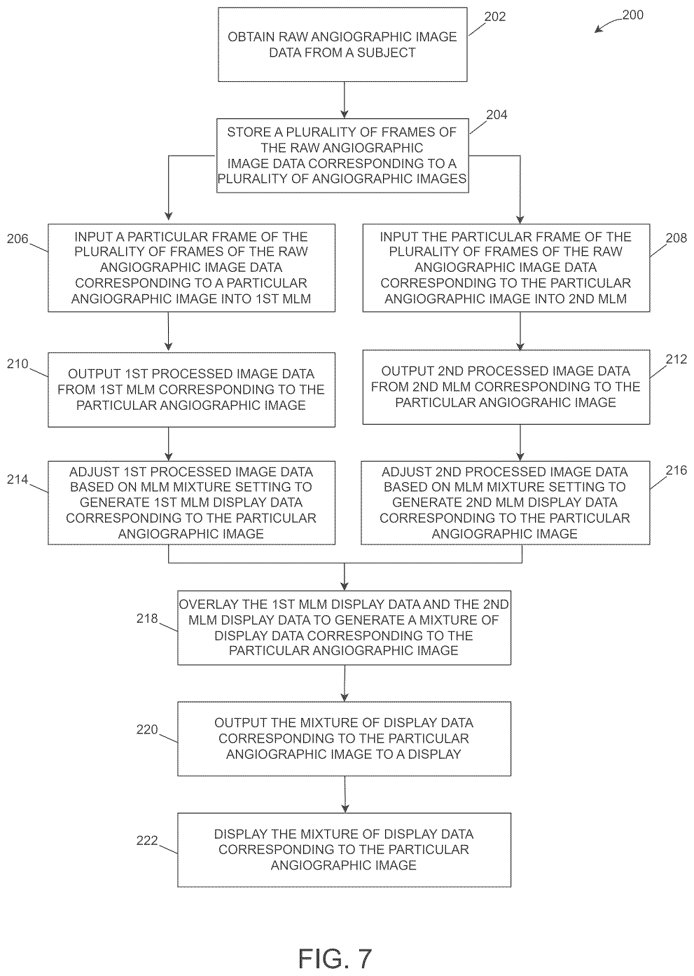

Referring to FIG. 4, the rotation of assemblies 32, 34 and the operation of the x-ray source are governed by a control mechanism 60 of the x-ray system. Control mechanism 60 includes an x-ray controller 62 that provides power and timing signals to x-ray source assembly 32. A data acquisition system (DAS) 64 in control mechanism 60 samples data from detector elements and passes the raw image data to an image reconstructor 65. Image reconstructor 65 receives digitized x-ray data from DAS 64 and may perform high speed image reconstruction according to the methods of the present disclosure. The reconstructed image is applied as an input to a computer 66 which stores the image in a mass storage device 69 or processes the image further. Image reconstructor 65 may be a standalone computer or may be integrated with computer 66. Image reconstructor 65 and/or computer 66 may include various modules, including neural network models, storage, and processors which together function to perform the various functionalities and methodologies described herein, including the methodology described below with respect to FIG. 7. Additionally or alternatively, image reconstructor 65 and computer 66 may operate in communication and/or cooperation with additional systems, such as information processing device 80, described below with respect to FIG. 5, and/or system 600, described below with respect to FIG. 6.

Control mechanism 60 also includes gantry motor controller 67 and a C-axis motor controller 68. In response to motion commands from computer 66, motor controllers 67, 68 provide power to motors in the x-ray system that produce the rotations about horizontal pivot axis 46 and C-axis 48, respectively. Computer 66 also receives commands and scanning parameters from an operator via operator console 70 that has a keyboard and other manually operable controls. An associated display 72 allows the operator to observe the reconstructed image frames and other data from computer 66. The operator supplied commands are used by computer 66 under the direction of stored programs to provide control signals and information to DAS 64, x-ray controller 62 and motor controllers 67, 68. In addition, computer 66 operates a table motor controller 74 which controls motorized table 36 to position the patient with respect to system isocenter 56.

Referring now to FIG. 5, a block diagram of a computer system or information processing device 80 (e.g., image reconstructor 65 and/or computer 66 in FIG. 4) is illustrated that may be incorporated into an angiographic imaging system, such as rotational x-ray system 28 of FIGS. 3 and 4, to provide enhanced functionality or to be used as a standalone device for visualization of angiographic data according to the various aspects and embodiments of the present invention described herein. Information processing device 80 may be local to or remote from rotational x-ray system 28.

In one example, the functionality performed by information processing device 80 may be offered as a Software-as-a-Service (SaaS) option. SaaS refers to a software application that is stored in one or more remote servers (e.g., in the cloud) and provides one or more services (e.g., angiographic image processing) to remote users. In one embodiment, computer system 80 includes a monitor or display 82, computer system 84 (which includes processor(s) 86, bus subsystem 88, memory subsystem 90, and disk subsystem 92), user output devices 94, user input devices 96, and communications interface 98. Monitor 82 can include hardware and/or software elements configured to generate visual representations or displays of information. Some examples of monitor 82 may include familiar display devices, such as a television monitor, a cathode ray tube (CRT), a liquid crystal display (LCD), a display panel, or the like. In some embodiments, monitor 82 may provide an input interface, such as incorporating touch screen technologies.

Computer system 84 can include familiar computer components, such as one or more central processing units (CPUs), memories or storage devices, graphics processing units (GPUs), communication systems, interface cards, or the like. As shown in FIG. 5, computer system 84 may include one or more processor(s) 86 that communicate with a number of peripheral devices via bus subsystem 88. Processor(s) 86 may include commercially available central processing units or the like. Bus subsystem 88 can include mechanisms for letting the various components and subsystems of computer system 84 communicate with each other as intended. Although bus subsystem 88 is shown schematically as a single bus, alternative embodiments of the bus subsystem may utilize multiple bus subsystems. Peripheral devices that communicate with processor(s) 86 may include memory subsystem 90, disk subsystem 92, user output devices 94, user input devices 96, communications interface 98, or the like.

Processor(s) 86 may be implemented using one or more analog and/or digital electrical or electronic components, and may include a microprocessor, a microcontroller, an application-specific integrated circuit (ASIC), a field programmable gate array (FPGA), programmable logic and/or other analog and/or digital circuit elements configured to perform various functions described herein, such as by executing instructions stored in memory subsystem 90 and/or disk subsystem 92 or another computer program product.

Memory subsystem 90 and disk subsystem 92 are examples of physical (non-transitory) storage media configured to store data. Memory subsystem 90 may include a number of memories including random access memory (RAM) for volatile storage of program code, instructions, and data during program execution and read only memory (ROM) in which fixed program code, instructions, and data are stored. Disk subsystem 92 may include a number of file storage systems providing persistent (non-volatile) storage for programs and data. Other types of physical storage media include floppy disks, removable hard disks, optical storage media such as compact disc-read-only memories (CD-ROMS), digital video disc (DVDs) and bar codes, semiconductor memories such as flash memories, read-only-memories (ROMS), battery-backed volatile memories, networked storage devices, or the like.

Memory subsystem 90 and disk subsystem 92 may be configured to store programming and data constructs that provide functionality or features of techniques discussed herein, including, for example, software code modules which implement the machine learning models described herein, and/or processor instructions that, when executed by processor(s) 86, implement or otherwise provide the various functionalities and methods described herein. Such programming and data constructs may be stored in memory subsystem 90 and disk subsystem 92. Memory subsystem 90 may be a non-transitory computer readable storage medium.

User input devices 96 can include hardware (physical) and/or software (graphical) control elements configured to receive input from a user for processing by components of computer system 80. User input devices can include all possible types of devices and mechanisms for inputting information to computer system 84. These may include a keyboard, a keypad, a touch screen, a touch interface incorporated into a display, audio input devices such as microphones and voice recognition systems, and/or other types of input devices. In various embodiments, user input devices 96 may include a computer mouse, a trackball, a track pad, a joystick, a wireless remote, a drawing tablet, a voice command system, an eye tracking system, or the like. In some embodiments, user input devices 96 are configured to allow a user to select or otherwise interact with objects, icons, text, or the like that may appear on monitor 82 via a command, motions, or gestures, such as a click of a button or the like.

User output devices 94 can include hardware and/or software elements configured to output information to a user from components of computer system 80. User output devices can include all possible types of devices and mechanisms for outputting information from computer system 84. These may include a display (e.g., monitor 82), a printer, a touch or force-feedback device, audio output devices, or the like.

Communications interface 98 can include hardware and/or software elements configured to provide unidirectional or bidirectional communication with other devices. For example, communications interface 98 may provide an interface between computer system 84 and other communication networks and devices, such as via an internet connection.

In one example, information processing device 80 may obtain (e.g., via communications interface 98) a series of angiographic image frames (e.g., an angiogram) at a rate faster than cardiac frequency. For instance, information processing device 80 may use wavelet angiography (e.g., by employing complex-valued wavelet transforms) to generate a spatiotemporal reconstruction of cardiac frequency phenomena in an angiogram obtained at faster than cardiac frequency. Spatiotemporal reconstruction may be performed in accordance with techniques described in U.S. Pat. No. 10,123,761, filed Jul. 1, 2016, issued Nov. 13, 2018, and titled DEVICE AND METHOD FOR SPATIOTEMPORAL RECONSTRUCTION OF A MOVING VASCULAR PULSE WAVE IN THE BRAIN AND OTHER ORGANS, which is hereby incorporated by reference herein in its entirety.

Referring to FIG. 6, an example computing environment 100 is shown for executing the functions and methodologies of the various embodiments of the invention described herein. Computing environment 100 may include a server 135 in communication with, via network 154, one or more of a client computer system 160, an imaging device 158, and a database 156. Server 135 and client computer system 160 may each include computer system/information processing device 80, described above with respect to FIG. 5, or may include a different computer system. Imaging device 158 may include an angiogram imaging system in the form of rotational x-ray system 28 described above with respect to FIGS. 3 and 4, one or more components thereof, and/or a different imaging device. Database 156 may be remotely configured relative to server 135 of system 100, and in communication therewith via network 154.

Network 154 may include a local area network (LAN), a wide area network (WAN) such as the Internet, or a combination of the two, and include wired, wireless, or fiber optic connections. In general, network 154 can be any combination of connections and protocols known in the art that will support communications between server 135 and database 156, client computer system 160, and imaging device 158 via their respective network interfaces.

It will be understood that the functional division among components of computing system 100 are not to be construed as a limiting example. In certain embodiments, system server 135 of computing system 100 includes a network interface (I/F) 136, a video controller 138, memory 140, one or more databases/storage 155, and at least one processor 137 in communication with network interface (I/F) 136, video controller 138, memory 140, and database 155. System 100 may also include or be in communication with a display 142 in operative communication with video controller 138 for displaying images, and a user input device 144 configured to provide one or more user inputs to processor 137. In certain embodiments, memory 140 comprises a non-transitory computer readable medium that stores instructions executable by the processor 137 to perform the functions and methodologies of the various embodiments of the invention described herein. For example, memory 140 may store a first machine learning model (Mₚ) 146, a second machine learning model (Mₛ) 148, an image generator module 150 for processing and adjusting image data in accordance with outputs from first and second machine learning models 146, 148 and inputs from user input device 144, and a graphical user interface module 152 configured to display a graphical user interface via video controller 138 and display 142. Graphical user interface module 152 may be configured to, for example, provide the various graphical user interfaces described above with respect to FIGS. 1A-2C.

As described above, first machine learning model Mₚ 146 may be a deep learning neural network model trained and optimized for high specificity to vessel pixels, and second machine learning model Mₛ 148 may be a deep learning neural network model trained and optimized for high sensitivity. User input device 144 may include a laptop computer, a tablet computer, a netbook computer, a personal computer (PC), a desktop computer, a personal digital assistant (PDA), a smart phone, a thin client, or any programmable electronic device capable of executing computer readable program instructions.

Each of the modules and models stored in memory 140 of server 135 may include one or more sub modules or models to perform various example functions and methodologies of the present invention, be implemented by any combination of any quantity of software and/or hardware modules or units, and reside within memory 140 of server 135 for execution by a processor, such as processor 137.

Database 155 and remote database 156 may include any non-volatile storage media known in the art. For example, databases 155, 156 can be implemented with a tape library, optical library, one or more independent hard disk drives, or multiple hard disk drives in a redundant array of independent disks (RAID). Similarly, image data in databases 155, 156 may conform to any suitable storage architecture known in the art, such as a file, a relational database, an object-oriented database, and/or one or more tables. The above described system and/or components thereof may be used to implement various methodologies of the present invention described herein, including method 200, described below with respect to FIG. 7.

Each element in flowcharts shown or methodologies described herein, such as process 200 shown and described below with respect to FIG. 7, depicts a step or a group of steps of novel computer-implemented systems and methodologies for displaying an angiographic image generated from multiple deep learning models in concert with one or more pre-set or user-adjustable parameters. Each step of methodologies described herein may contain one or more sub-steps. For purposes of illustration and explanation, these steps, as well as all other steps identified and described, are presented in a certain logical order. However, it will be appreciated that any exemplary embodiments described herein can contain an alternate order of the steps adapted to a particular application of a technique disclosed, and that any such variations and/or modifications are intended to fall within the scope of the invention. The depiction and description of steps in any particular order is not intended to exclude embodiments having the steps in a different order, unless required by a particular application, explicitly stated, or otherwise clear from the context.

Referring to FIG. 7, process 200 illustrates an example methodology that may be implemented by computing environment 100 and/or computer/information processing device 80 and/or any other suitable computing system. As shown, at Step 202, the server 135 obtains, via processor 137, raw angiographic image data of a subject from an angiographic imaging device. In certain embodiments, the processor 137 may obtain the raw angiographic data directly from rotational x-ray system 28, from client computer system 160, remote database 156, or from imaging device 158 via network 154. In other embodiments, server 135 may be implemented as part of an imaging device, and obtain the raw angiographic image data directly via the imaging device during an angiogram conducted on the subject.

At Step 204, processor 137 stores a plurality of frames of the raw angiographic image data corresponding to a plurality of angiographic images in database 155.

At Step 206, processor 137 inputs a particular frame of the plurality of frames of the raw angiographic image data corresponding to a particular angiographic image into first machine learning model (Mₚ) 146, which has been trained with a loss function optimized for high specificity. In certain embodiments, processor 137 may retrieve the particular frame from database 155. As described above with respect to FIGS. 1A-2C, the particular frame corresponding to the particular angiographic image from the angiogram or angiographic study may be selected by a user via a control element, such as graphical user control element 106a or 206a, which the user may manipulate (e.g., via a horizontal scroll bar) on a graphical user interface of a display, such as display 142 or a display of user input device 144, to select a particular frame. Alternatively, the system may be configured to preselect a particular frame from the angiogram, and/or to iterate through the entire plurality of image frames in sequence, which the user may or may not select for further study/operations. At Step 208, processor 137 similarly inputs the particular frame into second machine learning model (Mₛ) 148, which has been trained with a loss function optimized for high sensitivity.

At Step 210, the first machine learning model (Mₚ) 146 outputs first image data of high specificity to vessel pixels as described above. At Step 212, second machine learning model (Mₛ) outputs second image data of high sensitivity to vessel pixels as described above.

It will be appreciated that in certain embodiments, Steps 206-212 may be performed by processor 137 for the plurality of frames of the angiogram when the raw angiographic image data is obtained and stored at steps 202-204, not just for a particular frame. For example, in certain embodiments, processor 137 may be instructed to input all frames of the raw angiographic data to the first and second machine learning models, and to store first and second processed image data outputted from the first and second machine learning models for each frame in database 155. Alternatively, to save space and energy, system 100 may be configured to await selection of a particular frame by a user, or may be preset to analyze each frame sequentially upon approval by a user.

At Steps 214, 216 processor 137 adjusts the first image data and the second image data based on a machine learning model mixture setting to generate first machine learning model display data and second machine learning model display data, respectively, both corresponding to the particular angiographic image. As described above, a mixture of the raw image with model-generated images may be controlled by the user via a raw versus model-generated selector control element, such as control elements 106b and 206b in FIGS. 1 and 2. Furthermore, as described above, a mixture of the model-generated image data may be controlled by the user via a model mixing control element, such as control element 106c in FIG. 1 and control element 106d in FIG. 2. The position of the model mixing control element governs the machine learning model mixture (e.g., the weight or contribution of each machine learning model Mₚ, Mₛ to the displayed image). In certain embodiments and methodologies, such as that contemplated in FIG. 7, no raw image data need be overlayed. Thus, only the first machine learning model display data and the second machine learning model display data are utilized for display.

In certain embodiments, the setting of the model mixing control element (or the general setting of the weights of the machine learning models) may be predetermined in system 100, and either adjustable or not adjustable by a user. In yet other embodiments, the mixture setting may be programmable, for example, or even recommended by system 100 based on a condition of the raw angiographic data of the particular angiographic frame.

At Step 218, processor 137 overlays the first machine learning model display data and the second machine learning display data to generate a mixture of display data corresponding to the particular angiographic image. At Steps 220, 222, processor 137 outputs and displays the mixture of display data corresponding to the particular angiographic image on a display in accordance with the various embodiments described above with respect to FIGS. 1A-2C. For example, processor may overlay the machine learning models by aligning their respective pixels in the high specificity and high sensitivity model-generated images and adjusting an opacity of the respective pixels as described herein.

In addition to acquiring angiographic images, additional cardiac signals/data may be contemporaneously acquired to serve as a cross correlation target. For example, the cardiac signals/data may serve as a reference cardiac signal for phase indexing pixels in the angiographic projections.

The above description is for the purpose of teaching the person of ordinary skill in the art how to practice the subject of the present application, and it is not intended to detail all those modifications and variations of it which will become apparent to the skilled worker upon reading the description. For example, while examples of a graphical user interface are described herein for combining outputs of one or more machine learning models trained as described herein (e.g., for specificity and sensitivity, respectively), it will be appreciated that the outputs may be displayed using other graphical user interfaces. It will also be appreciated that one or more machine learning models trained as described herein may be provided for integration into an imaging device to post-process raw image data from the imaging device, or as a standalone product (e.g., an image post-processing system or a software-as-a-service product) configured to receive raw image data and provide images with adjustable attributes, such as specificity and sensitivity. Furthermore, while techniques are described for combining the outputs of multiple machine learning models trained to optimize or accentuate different attributes (e.g., specificity and sensitivity) of angiographic images, it will be appreciated that such techniques may also be used to combine the outputs of multiple machine learning models trained to optimize or accentuate attributes of other types of images (e.g., X-ray images of other organs, CT-images, or MRI-images). It will additionally be appreciated that, instead of or in addition to neural network models, non-neural network image classifiers (such as Vision Transformers (ViT) and Support Vector Machines (SVMs)) may be used to generate images with desired attributes (such as one segmented image with high specificity and another segmented image with high specificity) from raw image data, and that such generated images may be combined and displayed as described herein. It is intended that all such modifications and variations be included within the scope of the present invention.

What is claimed:

1. A method for displaying angiographic images on a display connected to a computer, the method comprising:
obtaining, with the computer, a first angiographic image generated by a first machine learning model from angiographic data and a second angiographic image generated by a second machine learning model from the angiographic data, wherein the first machine learning model is configured to have greater sensitivity performance than the second machine learning model and wherein the second machine learning model is configured to have greater specificity performance than the first machine learning model; and
displaying, with the computer via a display, a mixture of the first and second angiographic images generated by the first and second machine learning models.

2. The method of claim 1, wherein the first angiographic image comprises a first set of pixels and the second angiographic image comprises a second set of pixels, and wherein displaying the mixture of the first and second angiographic images comprises superposing the first and second sets of pixels.

3. The method of claim 2, further comprising adjusting, via the computer, the mixture of the first and second angiographic images by increasing or decreasing an opacity of one of the first and second sets of pixels relative to an opacity of the other of the first and second sets of pixels.

4. The method of claim 3, further comprising receiving, with the computer, an input from a user, and adjusting the mixture of the first and second angiographic images based on the input from the user.

5. The method of claim 4, wherein:
the input from the user is received from a user-adjustable control element connected to the computer;
the control element has a range of adjustment; and
at one end of the range, an opacity of the first set of pixels is greater than an opacity of the second set of pixels, and at another end of the range, the opacity of the second set of pixels is greater than the opacity of the first set of pixels.

6. The method of claim 5, wherein the control element is a mechanical control element or a graphical control element.

7. The method of claim 6, wherein the control element is a graphical control element, and further comprising displaying, with the computer via the display, a graphical user interface comprising the graphical control element.

8. The method of claim 7, wherein the graphical control element is adjustable with a pointing device connected to the computer.

9. The method of claim 1, wherein the first and second machine learning models are neural network models, the first machine learning model is trained with a first loss function, and the second machine learning model is trained with a second loss function that is different than the first loss function in terms of sensitivity and specificity performance.

10. The method of claim 1, wherein displaying further comprises mixing the angiographic data with the first and second angiographic images generated by the first and second machine learning models.

11. The method of claim 10, further comprising adjusting, with the computer, a mixture of the angiographic data and the first and second angiographic images based on an input from a user received by the computer.

12. A method for displaying angiography images from machine learning models, the method comprising:
obtaining angiographic image data generated by utilizing two machine learning models with differing sensitivity and specificity performance;
providing a graphical user interface comprising a first graphical control element for controlling a mixture of the two machine learning models;
displaying an angiogram image based on a setting of the first graphical control element; and
adjusting the displayed image in response to a change in the setting of the first graphical control element.

13. The method of claim 12, wherein the graphical user interface further comprises a second graphical control element that offers user adjustment of a mixture between the two machine learning models and raw angiographic image data displayed in the displayed image.

14. The method of claim 13, wherein the graphical user interface further comprises a third graphical control element that offers user adjustment of a zoom setting, wherein adjusting the zoom setting simultaneously increases a magnification of the displayed image and a proportional mixture of a high sensitivity machine learning model in the displayed image.

15. The method of claim 14, wherein the second graphical control element controls a mixture of raw image data and a combined output of the machine learning models, in the displayed image.

16. A system comprising:
a computer with one or more processors;
a display; and
a non-transitory computer-readable medium coupled to the one or more processors and storing instructions thereon that, when executed by the one or more processors, causes the one or more processors to:
obtain a first angiographic image generated by a first machine learning model from angiographic data and a second angiographic image generated by a second machine learning model from the angiographic data, wherein the first machine learning model is configured to have greater sensitivity performance than the second machine learning model and the second machine learning model is configured to have greater specificity performance than the first machine learning model; and
display, on the display, a mixture of the first and second angiographic images generated by the first and second machine learning models.

17. The system according to claim 16, wherein the first angiographic image comprises a first set of pixels and the second angiographic image comprises a second set of pixels, and wherein displaying the mixture of the first and second angiographic images comprises superposing the first and second sets of pixels.

18. The system according to claim 17, wherein the instructions, when executed by the one or more processors, further cause the one or more processors to adjust the mixture of the first and second angiographic images by increasing or decreasing an opacity of one of the first and second sets of pixels relative to an opacity of the other of the first and second sets of pixels.

19. The system according to claim 17, wherein the instructions, when executed by the one or more processors, further cause the one or more processors to:
receive an input from a user; and
adjust the mixture of the first and second angiographic images based on the input from the user.

20. The system according to claim 19, wherein the input from the user is received from a user-adjustable control element connected to the computer, the control element has a range of adjustment, at one end of the range, an opacity of the first set of pixels is greater than an opacity of the second set of pixels, and at another end of the range, the opacity of the second set of pixels is greater than the opacity of the first set of pixels.

21. A computer program product comprising a non-transitory computer-readable medium storing instructions thereon that, when executed by one or more processors, causes the one or more processors to obtain a first angiographic image generated by a first machine learning model from angiographic data and a second angiographic image generated by a second machine learning model from the angiographic data, wherein the first machine learning model is configured to have greater sensitivity performance than the second machine learning model and the second machine learning model is configured to have greater specificity performance than the first machine learning model; and
display, on the display, a mixture of the first and second angiographic images generated by the first and second machine learning models.

22. The computer program product according to claim 21, wherein the first angiographic image comprises a first set of pixels and the second angiographic image comprises a second set of pixels, and wherein displaying the mixture of the first and second angiographic images comprises superposing the first and second sets of pixels.

23. The computer program product according to claim 22, wherein the instructions, when executed by the one or more processors, further cause the one or more processors to adjust the mixture of the first and second angiographic images by increasing or decreasing an opacity of one of the first and second sets of pixels relative to an opacity of the other of the first and second sets of pixels.

24. The computer program product according to claim 22, wherein the instructions, when executed by the one or more processors, further cause the one or more processors to:
receive an input from a user; and
adjust the mixture of the first and second angiographic images based on the input from the user.

25. The system according to claim 24, wherein the input from the user is received from a user-adjustable control element in communication with the one or more processors, the control element has a range of adjustment, at one end of the range, an opacity of the first set of pixels is greater than an opacity of the second set of pixels, and at another end of the range, the opacity of the second set of pixels is greater than the opacity of the first set of pixels.

26. A computer-implemented method for generating an angiographic image for use on a display connected to a computer, the method comprising:
obtaining raw angiographic image data from an angiogram of a subject;
storing, in a memory, a plurality of frames of the raw angiographic image data, the plurality of frames corresponding to a plurality of angiographic images;
inputting a particular frame of a plurality of frames of the raw angiographic data into a first machine learning model and a second machine learning model, wherein the first machine learning model is configured to have greater sensitivity performance than the second machine learning model and the second machine learning model is configured to have greater specificity performance than the first machine learning model;
outputting, from the first machine learning model, first processed image data corresponding to the particular angiographic image;
outputting, from the second machine learning model, second processed image data corresponding to the particular angiographic image;
adjusting, based on a model mixture setting set on the computer, the first processed image data and the second processed image data to generate first model display data and second model display data;
overlaying the first model display data and the second model display data to generate a mixture of display data corresponding to the particular angiographic image; and
outputting, for display, the output of the mixture of display data.

27. The computer-implemented method of claim 26, wherein the model mixture setting is user adjustable to change the mixture of display data.

28. The computer-implemented method of claim 27, further comprising:
adjusting the model mixture setting, based on an input from a user, by increasing or decreasing an opacity of at least one set of pixels corresponding to the first processed image data and/or the second processed image data.

29. The computer-implemented method of claim 28, wherein the input from the user is received from a user-adjustable control element connected to the computer.

30. The computer-implemented method of claim 29, wherein the control element has a range of adjustment, at one end of the range, an opacity of the first set of pixels is greater than an opacity of the second set of pixels, and at another end of the range, the opacity of the second set of pixels is greater than the opacity of the first set of pixels.
